(19) United States
(12) Patent Application Publication
(10) Pub. No.: US 2020/0046624 A1
(43) Pub. Date: Feb. 13, 2020

(54) FORMULATION FOR NECK DERMAL, EPIDERMAL AND SUBDERMAL INTEGRITY

(71) Applicant: Goodier Cosmetics, Dallas, TX (US)

(72) Inventors: Tatiana Kelly, Westlake, TX (US); Rita Cabrera, Idaho Falls, ID (US); Theresa Fox, The Colony, TX (US); John Koch, Fort Worth, TX (US); John Muller, Dallas, TX (US)

(21) Appl. No.: 16/100,038

(22) Filed: Aug. 9, 2018

(51) Int. Cl. A61K 8/64; A61K 8/37; A61K 8/365; A61K 8/67; A61K 8/58; A61K 8/34; A61K 8/06; A61Q 19/08

(52) U.S. Cl. CPC A61K 8/64; A61K 8/375; A61K 8/365; A61K 8/67; A61K 2800/434; A61K 8/347; A61K 8/06; A61Q 19/08; A61K 8/585

(57) ABSTRACT

In an exemplary embodiment there is presented a formulation that is topically applied to the outer jawline and neck skin to improve the appearance of the skin by repairing or concealing age-related skin effects, such as wrinkles, improving skin tone by reducing stretching and sagging of the skin, adding moisture to reduce skin dryness, and adding an appearance of brightness. An exemplary embodiment of the formulation includes a blend of oligosaccharides, medium chain triglycerides, peptides, silanol, and diglucosyl gallic acid, all provided in a viscous liquid emulsion base for ease of dispensing from a pump dispenser.

### FIELD OF THE INVENTION

The inventive technology relates to the field of dermal cosmetics, and more particularly to topical formulations the restoration of denial, epidermal and subdermal integrity in the jawline and neck region.

### DESCRIPTION OF THE RELATED ART

As a natural part of the ageing process, skin gradually loses its “tone” and wrinkles begin to emerge. The gradual loss of tone and increase in wrinkles varies considerably and in some the onset is earlier than in others. Many factors contribute to this loss of tone, some being genetic, and others a result of activities, such as exposure to higher levels of solar radiation from living in sunny climates, and environmental conditions. Many of the factors are outside of the immediate control of the individual.

There have been many efforts to arrest the gradual loss of skin tone brought about by age, and a significant sector of the health, surgery, nutraceuticals and cosmetics markets are directed to this goal. The products developed for topical application are specialized in that some are directed to the skin around the eyes, others to the facial area of the skin generally and others to the hands, and yet others are directed to the skin of the neck. The skin of the neck is, in general, relatively thin and not as well supported against gravity as in some other parts of the body. In addition, the skin is subject to gravitational pull that over time might be expected to cause gradual stretching. Taken together with ageing and resultant loss of elasticity and tone, the skin around the neck is susceptible to sagging and wrinkling. With modem fashions the area around the neck is often exposed. Accordingly, it is desirable address the issue.

While there are surgical techniques, and laser-based techniques for addressing the sagging and wrinkling of neck skin tissue, many people would prefer a topically applied formulation rather than undergo these procedures. Because of this need for non-invasive, non-surgical treatments, efforts have been made to address the stretching, sagging and wrinkling of skin around the neck region. An example of an effective formulation is the Nectifirm® (a registered trademark of Revision Corp., Inc. of Dallas, Tex.) formulation that is only available through physicians. The ingredients of this product, as disclosed online, includes a range of components, such as Palmitoyl Tripeptide-5, intended to reduce the appearance of fine lines and wrinkles. It also includes Faex (Baker's Yeast), Palmitoyl Hexapeptide-12 and Palmitoyl Tetrapeptide-7 to promoting an appearance of firmer lifted skin. Algae extracts are added to this formulation for skin moisture retention and tone. Likewise, Ceramide 2 enhances water retention and reduces skin dryness. To smooth the skin and reduce the appearance of fine lines and wrinkles, the formulation also has Bamboo, English Pea Extract and Glucosamine, Dihydromyricetin (from Wax Myrtle Shrub) reduces adipose tissue thereby providing a slimming appearance. In addition, Vitamins C and E are added to help skin appear brighter and more even toned. These ingredients are not the only ones in the formulation, but are represented as the main ingredients and may be assumed to be the active ingredients.

### SUMMARY

In an exemplary embodiment there is presented a non-allergenic formulation for jawline and neck dermal integrity, and sub-dermal integrity, that is topically applied to the outer surface of the skin, especially jawline and neck skin, to improve the appearance of the skin by repairing or concealing age-related skin effects, such as wrinkles and fine lines, improving skin tone by reducing stretching and sagging of the skin, adding moisture to reduce skin dryness, and adding an appearance of brightness. The formulation tightens and firms skin in the neck area, where there has been loss of viscoelasticity resulting in sagging and wrinkling. An exemplary embodiment of the formulation for jawline and neck dermal, epidermal and subdermal integrity includes a blend of: oligosaccharides, medium chain triglycerides, peptides, diglucosyl gallic acid, and silanol all provided in an emulsion base. Optionally, other components may be added to further enhance particular effects.

In an exemplary embodiment of the formulation for jawline and neck dermal, epidermal and subdermal integrity the oligosaccharides are present in the range from about 0.01 to about 10 wt. %.

In another exemplary embodiment of the formulation for jawline and neck dermal, epidermal and subdermal integrity the medium chain triglycerides are present in the range from about 0.01 to about 15 wt. %

In another exemplary embodiment of the formulation for jawline dermal, epidermal, subdermal and neck integrity wherein the peptides are present in the range from about 0.00002 to about 0.5 wt. %.

In another exemplary embodiment of the formulation for jawline and neck dermal, epidermal and subdermal integrity the polyphenols are present in the range from about 0.001 to about 5 wt. %.

In another exemplary embodiment of the formulation for jawline and neck dermal, epidermal and subdermal integrity the medium chain triglycerides have a fatty acid chain length from about 6 to about 12 carbons,

Optional additional components include, but are not limited to, from about 0.001 to about 2 wt. % ceramides; from about 0.001 to about 5 wt. % proteins; or from about 0.0001 to about 6 wt. % vitamins, including, but not limited to, vitamin C, vitamin E, and vitamin A; and/or vitamin derivatives. Further optionally, growth factors may also be added in appropriate amounts. Further additional components are discussed here below

In an exemplary treatment method, the formulation for jawline and neck dermal, epidermal and subdermal integrity is applied twice per day (morning and night) as a viscous lotion from a pump dispenser, or other dispenser, for a period of a few weeks to obtain results. The formulation is stable, and absorbs readily into the epidermis.

### BRIEF DESCRIPTION OF THE DRAWINGS

The foregoing aspects and many of the attendant advantages, of the present technology will become more readily appreciated by reference to the following Detailed Description, when taken in conjunction with the accompanying simplified drawings of exemplary embodiments. The drawings, briefly described here below, are not to scale, are presented for ease explanation of and do not limit the scope of the inventions recited in the accompanying patent claims.

FIG. 1 is a color photo of the jawline and neck of a representative person before treatment with an embodiment of the formulation for jawline and neck dermal, epidermal and subdermal integrity.

FIG. 2 is a color photo, for comparison with FIG. 1, of the jawline and neck of the same person as in FIG. 1, after treatment with an embodiment of the present formulation for jawline and neck dermal, epidermal and subdermal integrity.

### DETAILED DESCRIPTION

The patent or application file contains at least one drawing executed in color. Copies of this patent or patent application publication with color drawing(s) will be provided by the Office upon request and payment of the necessary fee.

The following non-limiting detailed descriptions of examples of embodiments of the invention may refer to appended Figure drawings and are not limited to the drawings, which are merely presented for enhancing explanations of features of the technology. In addition, the detailed descriptions may refer to particular terms of art, some of which are defined herein, as appropriate and necessary for clarity.

As used herein the term “wt. % of the formulation,” means weight percent based on the total weight (mass) of the active components, such as the oligosaccharide, the peptides, etc. as well as the weight (mass) of the emulsion base into which the active components are blended.

In an exemplary embodiment there is presented a formulation for jawline and neck dermal integrity, and sub-dermal integrity, that is topically applied to the outer surface of the skin, especially jawline and neck skin, to improve the appearance of the skin by repairing or concealing age-related skin effects, such as wrinkles and fine lines, “crepe-y skin,” improving skin tone by reducing stretching and sagging of the skin, adding moisture (and/or reducing moisture loss) to reduce skin dryness, and adding an appearance of brightness. The formulation tightens and firms skin in the neck area, where there has been loss of viscoelasticity resulting in sagging and wrinkling.

An exemplary embodiment of the formulation for jawline and neck dermal, epidermal and subdermal integrity includes a blend of: oligosaccharides, medium chain triglycerides, peptides, and diglucosyl gallic acid, all provided in an emulsion base. As detailed herein, other chemical components may be added to the formulation.

In general it has become increasingly apparent to the present inventors that the skin microbiome of the neck and jawline is of significance, and the present formulation has been developed with that microbiome in mind. In one aspect of the technology, the formulation includes peptides that can be classified based on functional type, such neck relaxant/neuro modulators and elastin modulation peptides, as explained in more detail here below.

It has also been found that the human skin is veiled with massive amounts of microorganisms (the skin microbiome) which live in a beneficial symbiosis with the skin. It is the goal to avoid disruption of the microbiome to maintain the health of the skin. It has been found that use of glucoside compounds tend to support or enrich the microbiome. Accordingly, in another aspect of the formulation for jaw line and neck dermal and sub-dermal integrity a component is included that converts to and releases gallic acid upon exposure on the skin to the skin biome. The microbiome will enzymatically convert the chemical into Tri Hydroxy Benzoic Acid (THBA), a skin active component to inhibit tyrosinase thereby resulting in uniform skin coloration. The diglucosyl gallic acid may be present in the formulation for jaw line and neck dermal and sub-dermal integrity an amount from 0.1 to 3 wt. %, or 0.1 to 2 wt. %.

As indicated above, an exemplary formulation for jawline and neck dermal integrity and sub-dermal integrity includes oligosaccharides, medium chain length triglycerides, diglucosyl gallic acid and peptides. The formulation may also include polyphenols, ceramides, silanol, sulfated galactofucan, flavonoids and/or phenylflavonoids, steroid saponins, flavonoid glycosides, vitamins, and preservatives. The formulation may be non-allergenic, and may be fragrance-free and alpha-hydroxy acids-free. Each of these components of the formulation as well as others are discussed here below.

The oligosaccharides, which are film formers, include, but are not limited to, hydrolyzed manihot esculenta tuber extract, and porphyridium polysaccharide and the like. Hydrolyzed manihot esculenta tuber extract is a non-limiting example of a useful glucan biopolymer that produces a rapid tensor effect causing smoothing of wrinkles and increasing skin elasticity while decreasing sagging. Porphyridium polysaccharide has a similar effect. Accordingly, oligosaccharides that perform similarly are useful in the formulations.

The medium chain triglycerides include those having a medium chain length, which means from about 6 to about 12 carbon atoms chain length. Examples of a useful medium chain length triglyceride include, but are not limited to cocos nucifera fruit extract, nangai oil; and caprylic/capric triglyceride acid, which is metabolized and converted to essential fatty acids. The medium chain triglyceride is intended to reinforce the skin barrier function by normalizing epidermal lipids thereby causing increased skin hydration (or moisture retention) due to decreased trans-epidermal water loss.

The peptides of the formulation are of several different types by functionality. For example, the peptides that increase collagen production (improve skin firmness) include, without limitation:
Palmitoyl Tripeptide-5
Palmitoyl Tripeptide-38
Palmitoyl Dipeptide-5 Diaminobutyroyl Hydroxythreonine
Tetradecyl Aminobutyroylvalylaminobutyric Urea Trifluoroacetate

The peptides that promote increased elastin synthesis (improve skin elasticity), include without limitation:
Acetyl Tetrapeptide-2
Trifluoroacetyl Tripeptide-2

The neuromodulating (ease muscle tension to prevent expression lines) peptides include without limitation: Acetyl Dipeptide-1 Cetyl Ester.

The peptides that facilitate cellular movement (optimize proliferation mechanisms) include, without limitation: Palmitoyl Hexapeptide-12.

From the foregoing and in addition thereto, each peptide in the range of useful peptides may facilitate a particular functionality of the formulation. For example, and without limitation, palmitoyl tripeptide-5, palmitoyl dipeptide-5 diaminobutyroyl hydroxytheonine and palmitoyl tripeptide-38 are peptides that increase the rate of collagen formation in the skin, and facilitate improved skin firmness and smoothness. Palmitoyl Tripeptide-38 enhances the synthesis of proteins (Collagen IV, laminins and fibronectin) at the dermal-epidermal junction to improve loose, sagging skin, Another peptide, such as acetyl dipeptide-1 cetyl ester, for example, is a sequenced lipopeptide that assists in muscle relaxation in the dermal layers leading to prevention of wrinkle onset. A further non-limiting exemplary peptide, is a sequenced lipopeptide that in the formulation targets the stimulation of hyaluronic acid, collagen I, III and IV, laminins and fibronectin thereby producing a rejuvenating lifting effect, increased skin firmness and a reduction in wrinkles. Another exemplary peptide, without limitation, includes acetyl tetrapeptide-2 which has a function in the formulation of preventing or reducing skin sagging by enhancing the proper assembly of elastin and increasing the gene expression of collagen producing improved skin firmness. Another peptide, trifluoroacetyl tripeptide-2, on the hand, in addition to reducing skin sagging by decreasing elastase activity producing and improved skin firmness, also improves skin elasticity and viscoelasticity. The additional exemplary non-limiting peptide palmitoyl hexapeptide-12 facilitates cell movement to stimulate reconstruction of the dermis thereby producing improved skin firmness and elasticity. Another useful peptide includes, without limitation, tetradecyl aminobutyroylvaly amino butyric urea trifluoroacetate.

Exemplary embodiments of the formulation for jawline and neck dermal, epidermal and subdermal integrity may include at least one, and preferably more than one, more preferably 3 to 5 of the above listed of peptides, or its functional equivalent, since each peptide listed may have counterparts having the same or similar functionality. The listing of all these counterparts would be superfluous, and they are not provided for brevity, but they would be found by a person of ordinary skill in the art after having read this description. Preferably, a formulation for jawline and neck dermal integrity and sub-dermal integrity includes an appropriate efficacious quantity of several of the peptides from the ones listed here above by function, or the functional equivalent of the peptide that may not be listed. In addition, preferably but not necessarily, the formulation includes an appropriate efficacious quantity of at least one from each of the peptide functional types identified here above.

As indicated above, the formulation for jawline and neck dermal, epidermal and subdermal integrity may include a ceramide, which contributes to moisture retention in the skin, and facilitates and increases the rate of skin cell renewal.

An exemplary embodiment of the formulation for jawline and neck dermal, epidermal and subdermal integrity includes a silanol. A non-limiting example includes methylsila carboxyrnethyl theophylline alginate, and the like, that provides lipolytic activity improving the breakdown of triglycerides into free fatty acids, and glycerol producing slimming and regeneration of the connective tissue of the skin. The silanol is present in the present formulation in an amount from about 0.001 to about 5 wt. %.

The formulation for jawline and neck dermal integrity and sub-dermal integrity may include a sulphated galactofucan. A non-limiting example includes Undaria Pinnatifida Extract (a brown macro-algae) that in the formulation is target to the extracellular matrix (ECM) thereby improving organization of collagen, elastin and hyaluronic acid and also decreasing wrinkles and improving skin firmness.

The formulation for jawline and neck dermal, epidermal and subdermal integrity may include flavonoids and phenylpropanoids. These are exemplified by Vaccinium Vitis-Idaea Callus Culture Extract, and the like which prevents oxidative stress induced by IR radiation, softens UV induced inflammation in keratinocytes and restores antioxidant capacity of skin producing firmer skin.

The formulation for jawline and neck dermal, epidermal and subdermal integrity may also include flavonoids, exemplified by Astragalus Membranaceus Root Extract, and the like, which have lipolytic activity to improve breakdown of triglycerides into free fatty acids and glycerol producing slimming and regeneration of connective tissue.

The formulation for jawline and neck dermal, epidermal and subdermal integrity may further include steroid saponins, a non-limiting example of which is Ruscus Aculeatus (Butcherbroom) Root Extract (ruscogenins) and the like to improve skin capillary circulation for removal of fat accumulations therein and thereby provides a slimming appearance.

The formulation for jawline and neck dermal, epidermal and subdermal integrity may also include flavonoid glycosides, such as Solidago Virgaurea (Goldenrod) Extract, Citrus Medica Linionum (Lemon) Peel Extract (rutin, naringin, hesperidin) and the like to provide improved capillary circulation for removal of fat accumulations in the skin.

As pointed out above, an exemplary embodiment of the formulation for jawline and neck dermal, epidermal and subdermal integrity includes a blend of: oligosaccharides, medium chain triglycerides, peptides, silanol, and polyphenols, and diglucosyl gallic acid all provided in an emulsion base. In the exemplary embodiment, the oligosaccharides may be present in the formulation in the range from about 0.01 to about 10 wt. % of the formulation. The medium chain triglycerides may be present in the formulation in the range from about 0.01 to about 15 wt. %. The peptides may be present in the formulation in the range from about 0.00002 to about 0.5 wt. %. The silanol may be present in the formulation in the range from about 0.001 to about 5 wt. %. Polyphenols may be present from about 0.001 to about 5 wt. %. The formulation includes from about 0.1 to about 5 wt. % diglucosyl gallic acid.

In general, it is preferred but not essential that the formulation be free of any alpha-hydroxy acids.

In general, the formulation for jawline and neck dermal, epidermal and subdermal integrity may include appropriate amounts of growth factors.

In general, the formulation for jawline and neck dermal, epidermal and subdermal integrity may include proteins. Proteins may be classified, in the dermal treatment arts, as those encouraging cellular movement, and as those that are regarded as neuro-stimulating. Appropriate amounts of either or both of these types of proteins may be added.

In general, the formulation for jawline and neck dermal, epidermal and subdermal integrity may include retinoids.

In general, the formulation for jawline and neck dermal, epidermal and subdermal integrity includes a base that is a carrier for the active ingredients. The base is an emulsion of either water in oil, or oil in water, such that the formulation has a consistency of a lotion that can be massaged into the skin. In general, the base emulsion is from about 40 to about 95 wt. % of the formulation.

For results, the formulation for jawline and neck dermal, epidermal and subdermal integrity should be applied to the skin area to be treated, typically the jawline and neck, at least twice per day—in the morning and at bed time. The application should cover the skin area and the formulation should be gently massaged into the skin. The formulation is skin absorbent albeit that the oligosaccharides are film forming agents. Thus, a film will remain on the skin to protect the surface and reduce moisture loss from the surface. Active ingredients may absorb into the skin to perform their respective functions. Treatment should continue for at least 6 to 8 weeks for noticeable improvement.

### EXAMPLE

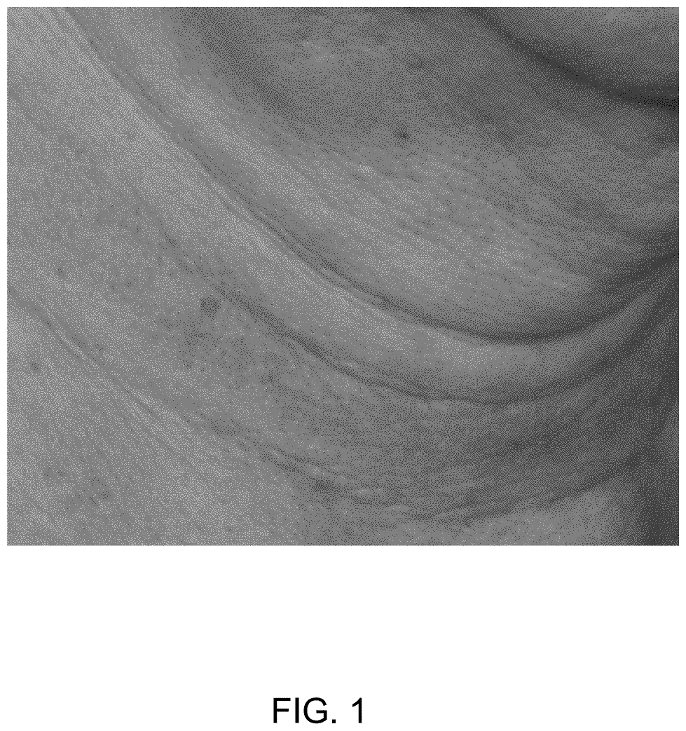

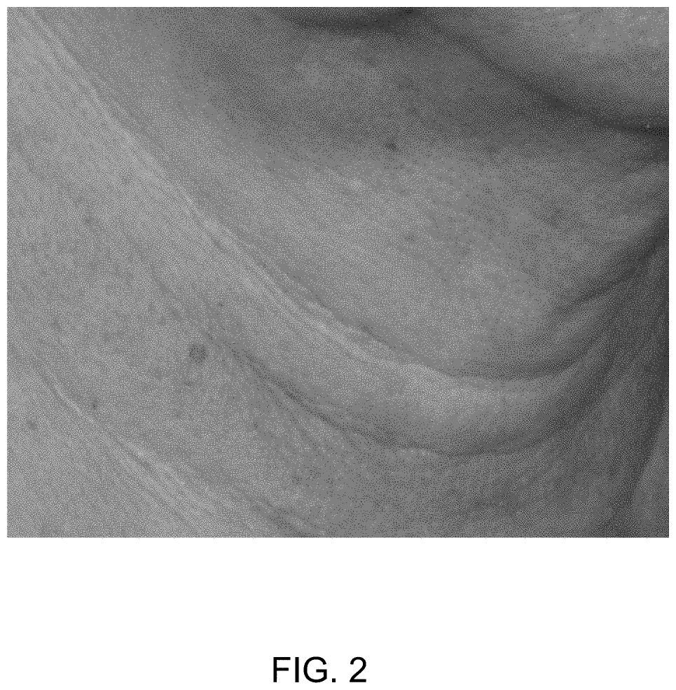

A test was carried out to demonstrate the efficacy of the formulation for jawline and neck dermal, epidermal and subdermal integrity of the present technology. The observed results indicated an improvement in the appearance of the subjects treated with the present formulation, as compared to the appearance before treatment especially in the jawline area but also in the neck region. This can be seen from FIG. 1 which shows an exemplary subject before treatment, while FIG. 2 shows the subject after treatment with the present formulation for jawline and neck dermal, epidermal and subdermal integrity.

While examples of embodiments of the technology have been presented and described in text and some examples also by way of illustration, it will be appreciated that various changes and modifications may be made in the described technology without departing from the scope of the inventions, which are set forth in and only limited by the scope of the appended patent claims, as properly interpreted and construed.

1. A topically applied cosmetic formulation for restoring age-related loss of neck dermal and subdermal integrity, the formulation comprising a blend of:
oligosaccharides, medium chain triglycerides, peptides, and diglucosyl gallic acid;
whereby daily application of the cosmetic formulation improves jawline skin and neck appearance by repairing or concealing wrinkles and improving skin tone by reducing stretching and sagging of the skin in areas where age-related loss of elasticity has occurred.

2. The formulation of claim 1, wherein the oligosaccharides are present from about 0.01 to about 10 wt. %.

3. The formulation of claim 1, wherein the medium chain triglycerides are present from about 0.01 to about 15 wt. %.

4. The formulation of claim 1, wherein the peptides are present from about 0.00002 to about 0.5 wt. %.

5. The formulation of claim 1, further comprising a silanol in an amount from about 0.001 to about 5 wt. %.

6. The formulation of claim 1, further comprising polyphenols in an amount from about 0.001 to about 5 wt. %.

7. The formulation of claim 1, further comprising growth factors.

8. The formulation of claim 1, further comprising vitamins.

10-21. (canceled)

22. The formulation of claim 1, wherein the medium chain triglycerides have a fatty acid chain length from about 6 to about 12 carbons.

23. The formulation of claim 1, comprising from 0.1 to 5 wt. % diglucosyl gallic acid.

24. The formulation of claim 1, wherein the peptides comprise a plurality of different peptides, the plurality of peptides each having from 2 to 100 amino acids.

25. The formulation of claim 24, wherein the peptides of the plurality of peptides are selected from peptides that promote collagen production, peptides that promote increased elastin, peptides that facilitate cellular motion, and peptides that are neuromodulating.

26. The formulation of claim 24, wherein the peptides are selected from the group consisting of: Palmitoyl Tripeptide-5, Palmitoyl Tripeptide-38, Palmitoyl Dipeptide-5 Diaminobutyroyl Hydroxythreonine, Tetradecyl Aminobutyroylvalylaminobutyric Urea Trifluoroacetate, Acetyl Tetrapeptide-2, Trifluoroacetyl Tripeptide-2, Acetyl Dipeptide-1 Cetyl Ester and Palmitoyl Hexapeptide-12.

27. The formulation of claim 1, wherein the oligosaccharides comprise a glucan biopolymer.

28. The formulation of claim 1, comprising an emulsion for ease of delivery to the skin via a pump dispenser.

29. The formulation of claim 1, further including any one or more of:
ceramides;
proteins;
vitamins; and
growth factors.

30. The formulation of claim 1, wherein the formulation is free of alpha-hydroxy acids.

31. A topically applied cosmetic formulation for restoring age-related loss of neck dermal and subdermal integrity, the formulation comprising a liquid blend of:
about 0.01 to about 10 wt.% oligosaccharides, medium chain triglycerides, peptides, and about 1 to about 5 wt. % diglucosyl gallic acid;
whereby twice daily application of the cosmetic formulation improves jawline skin and neck appearance by repairing or concealing wrinkles and improving skin tone by reducing stretching and sagging of the skin in areas where age-related loss of viscoelasticity has occurred.

32. The formulation of claim 31, wherein the blend comprises from about 0.00002 to about 0.5 wt. % peptides.

33. The formulation of claim 31, wherein the peptides are selected from the group consisting of: Palmitoyl Tripeptide-5, Palmitoyl Tripeptide-38, Palmitoyl Dipeptide-5 Diaminobutyroyl Hydroxythreonine, Tetradecyl Aminobutyroylvalylaminobutyric Urea Trifluoroacetate, Acetyl Tetrapeptide-2, Trifluoroacetyl Tripeptide-2, Acetyl Dipeptide-1 Cetyl Ester and Palmitoyl Hexapeptide-12.
